(19) United States
(12) Patent Application Publication
(10) Pub. No.: US 2025/0332118 A1
(43) Pub. Date: Oct. 30, 2025

(54) NOVEL ORAL CARE FORMULATIONS FOR TREATMENT OF XEROSTOMIA

(71) Applicant: LIVIONEX, Inc., Los Gatos, CA (US)

(72) Inventors: Rajiv BHUSHAN, Los Gatos, CA (US); Amit GOSWAMY, Los Gatos, CA (US)

(21) Appl. No.: 18/855,302

(22) PCT Filed: Apr. 8, 2023
(86) PCT No.: PCT/US2023/017987

§ 371 Date: Oct. 8, 2024

Related U.S. Application Data
(60) Provisional application No. 63/329,355, filed on Apr. 8, 2022.

(51) Int. Cl. A61K 31/047; A61K 8/34; A61K 9/00; A61K 31/10; A61K 31/198; A61K 47/02; A61K 47/24; A61K 47/36; A61P 1/02; A61Q 11/00

(52) U.S. Cl. CPC A61K 31/047; A61K 8/345; A61K 9/006; A61K 31/10; A61K 31/198; A61K 47/02; A61K 47/24; A61K 47/36; A61P 1/02; A61Q 11/00

(57) ABSTRACT

A formulation for treating xerostomia and alleviating the symptoms of dry mouth is provided. Formulations comprising a humectant (such as propylene glycol), a chelator (such as EDTA and its salts) and a transport enhancer (such as methylsulfonylmethane (MSM)) are provided.

### CROSS-REFERENCE TO RELATED APPLICATIONS

This application is a 371 U.S. National Stage filing of an international application filed under the Paris Convention Treaty (PCT) No. PCT/US2023/017987, titled “NOVEL ORAL CARE FORMULATIONS FOR TREATMENT OF XEROSTOMIA,” filed Apr. 8, 2023 which claims priority to U.S. Provisional Patent Application Ser. No. 63/329,355 titled “NOVEL ORAL CARE FORMULATIONS FOR TREATMENT OF XEROSTOMIA,” filed 8 Apr. 2022, the contents of which are incorporated herein in their entirety by reference.

### TECHNICAL FIELD OF THE INVENTION

This invention relates to the field of agents that treat xerostomia and alleviate the symptoms of dry mouth. In particular, the invention relates to oral care formulations containing a humectant, a transport enhancer, and a chelating agent. In one exemplary embodiment, it relates to oral care compositions containing MSM as a transport enhancer and chelators generally acceptable as safe (“GRAS”).

### BACKGROUND OF THE INVENTION

Xerostomia is a dry mouth condition caused by reduced or absent saliva flow. This condition can result in discomfort, interfere with speech and swallowing, make wearing dentures difficult, cause halitosis (halitosis), and impair oral hygiene by causing a decrease in oral pH and an increase in bacterial growth. Long-standing xerostomia can result in severe tooth decay (Caries) and oral candidiasis (chronic mucocutaneous candidiasis (CMC)). Xerostomia is a common complaint among older adults, affecting about 20% of the elderly.

Dry mouth is a growing problem. As a person ages, the number of medications taken by the person increases. About 2,000 medications are known to cause dry mouth. 75% of prescription drugs and many over-the-counter (OTC) drugs have dry mouth as a side effect. The most common include the following: anticholinergics, anti-Parkinsonian drugs, antineoplastics (chemotherapy), anti-hypertensives, anxiolytics, and antidepressants. As a result, about 70 million persons suffer from dry mouth and 1 out of 3 persons over the age of 45 have dry mouth.

Chemotherapy drugs cause severe dryness and stomatitis while they are being taken; these problems usually end after therapy is stopped. Other common drug classes that cause xerostomia include antihypertensives, anxiolytics, and antidepressants (less severe with selective serotonin reuptake inhibitors (SSRIs) than with tricyclics).

The rise of illicit methamphetamine use has resulted in an increasing incidence of meth mouth, which is severe tooth decay and periodontal inflammation caused by methamphetamine-induced xerostomia. The damage is exacerbated by the bruxing and clenching caused by the drug along with elevated intake of sugary beverages and poor oral hygiene while under the influence of the drug. This combination causes very rapid destruction of teeth. Tobacco uses also causes a decrease of saliva.

Several systemic disorders such as Sjögren's syndrome, diabetes mellitus, rheumatoid arthritis, and systemic lupus erythematosus are also known to cause oral dryness. Other causes include head and neck injuries which damage nerves to the salivary glands. Head and neck cancers in which radiation and chemotherapy are used is another cause of dry mouth. Tobacco use usually causes a decrease of saliva. The rise of illicit methamphetamine use has resulted in an increasing incidence of meth mouth, which is severe tooth decay caused by methamphetamine-induced xerostomia. The damage is exacerbated by the bruxing and clenching caused by the drug and by the heat of the inhaled vapor. This combination causes very rapid destruction of teeth.

With regard to an increasingly elderly population and its dependent use of medication, a multitude of negative side effects associated with disturbed salivary function will present a medico-socio-economic problem not only for the individual per se but also for society in the near future.

Problems with dry mouth include difficulty in swallowing and eating, difficulty in speaking because of dry vocal cords, the feeling of thirst, soreness of the throat and mouth, cracked lips, parched tongue and parched mouth. In addition to these physical problems, dry mouth results in oral health problems such as the increased risk for cavities, gingivitis (inflamed gums), periodontitis (inflammation of gums leading to loss of teeth), mouth sores, fungal infections of the mouth, and bad breath. It is thus important to know the degree of dryness of the mouth so that precautions can be taken to prevent oral health problems.

The cause of xerostomia is often apparent, but if the etiology is unclear and systemic disease is considered possible, further assessment should be pursued with a biopsy of a minor salivary gland (for detection of Sjögren syndrome, sarcoidosis, amyloidosis, TB, or cancer) and HIV testing. The lower lip is a convenient site for biopsy. Further information about xerostomia is available at www(dot)merckmanuals(dot)com/professional/dental-disorders/symptoms-of-dental-and-oral-disorders/xerostomia (accessed Apr. 2, 2022).

The lack of moisture and lubrication typically provided by saliva has a range of negative effects on oral tissue (soft tissue) ranging from mild discomfort to extremely painful and infected mouth sores. The persistent discomfort and dryness can also contribute to larger health issues by causing disruption of sleep, and impairing one's ability to talk (socialize, may impact psychological health) and cat (may impact nutrition). Dry buccal tissue is a less effective barrier and more susceptible to penetration by physical irritants such as toxins and carcinogens in foods, beverages and tobacco.

Saliva is also the mouth's primary defense against tooth decay. Healthy saliva flow helps prevent cavities by physically removing bacteria from the oral cavity before they can become attached to tooth and tissue surfaces and form a protected biofilm. The flow of saliva also helps dilute sugars and acids introduced by intake of food and beverages. The buffering capacity neutralizes acids and aids in the digestive process. The presence of calcium and phosphate salts provides continuous opportunity for re-mineralization of tooth enamel, serving to reverse the tooth decay process.

Many who suffer with xerostomia use separate products to address hard tissue health and soft tissue comfort. For soft tissue comfort, saliva substitute products are typically designed to provide lubrication and moisture. The format of these products is varied, and includes viscous gels/pastes, sprays, rinses, mints, and slow-release tablets. These are applied multiple-times per day or as needed for comfort. For hard-tissue health, different treatments are used to directly address cavity prevention (antiseptic rinses, fluoride products, calcium/phosphate treatments). Often “dry mouth-friendly” versions of products, such as toothpastes and mouth rinses are recommended. Dry mouth friendly products typically have a neutral pH and do not contain alcohol or other irritating components (e.g., anionic surfactants or emulsifiers).

Various oral care compositions, such as toothpastes and mouth-rinses, that are generally available, are based primarily on the cleaning abilities of surfactants, soaps, and or detergents along with mild abrasives. Some formulations have added antimicrobial agents, for example TRICLOSAN® or stannous fluoride to enhance antiplaque action.

It has recently been demonstrated that formulations comprising a transport enhancer (such as MSM) and a chelating agent (such as EDTA) showed dramatic reductions in plaque formation and have been used for prophylaxis and treatment of adverse oral conditions and disorders. (WO 2013/166459 by Bhushan et al.; WO 2014/100775 by Bhushan et al.; U.S. Pat. No. 9,616,008 by Bhushan et al.). A dental treatment product comprising MSM and a chelator in accordance with the disclosure of the above patent applications are available in the market as LIVIONEX® dental gel.

Humectants have been used in oral care products to prevent hardening of a toothpaste upon exposure to air, or in the case of a mouthwash, to provide improved moisturizing and mouthfeel and enhance the miscibility of poorly soluble components such a flavoring oils.

Propylene glycol is a small organic alcohol commonly used in toothpaste as a humectant. Propylene glycol has been used in dental care products such as toothpastes to ensure products remain free of unwanted bacteria, yeast and mold. (COLGATE®).

There is a desire to design a single product that effectively and easily addresses the need for dry mouth symptom relief (soft tissue comfort) and oral health preventative benefits (cavity protection).

### SUMMARY OF THE INVENTION

It is now observed that oral care products (disclosed in WO 2013/166459 by Bhushan et al.; WO 2014/100775 by Bhushan et al.; U.S. Pat. No. 9,616,008 by Bhushan et al.), comprising a transport enhancer (such as MSM) and a chelating agent (such as EDTA) showing dramatic reductions in plaque formation and used for prophylaxis and treatment of adverse oral conditions and disorders, demonstrated an unexpected and surprising effect of considerable and highly significant increases in unstimulated and stimulated saliva production, and alleviation of the feeling of dry mouth in subjects.

The instant invention provides a formulation comprising a humectant (such as humectant is selected from glycerol, sorbitol, xylitol, maltitol, propylene glycol, hexylene glycol, butylene glycol, erythritol, isomalt, and a combination thereof), a transport enhancer (such as MSM) and a chelating agent (such as EDTA).

The invention provides a formulation comprising: a humectant the GRAS (generally regarded as safe) list from the United States Food and Drug Administration (FDA), wherein the chelating agent is suitable for long-term consumption; a permeation enhancer that is methylsulfonylmethane (MSM); one or more inert excipients; and a liquid vehicle or carrier; wherein the chelating agent and the permeation enhancer are present in proportions effective to maintain homeostasis of iron levels in the body when consumed in regular doses, and wherein he percentage of the humectant is about 0.5% to about 15.0%, the percentage of chelator is about 0.0001% to 15% and the percentage of permeation enhancer in the composition is about 0.0001% to 0% by weight, respectively.

In one aspect of the invention, the formulation may be administered in any form suitable including paste, gel, solid and particulate solid-state compositions, such as kinds of toothpaste, gel dentifrices, tooth powders, chewing gums, tablets and lozenges. Additionally, in a particular embodiment, the formulation is entirely composed of components that are naturally occurring and/or classified as GRAS (“Generally Regarded as Safe”) by the U.S. Food and Drug Administration. However, the invention also contemplates non-GRAS components in the formulations.

In a particular embodiment, the humectant is propylene glycol.

The method involves administering to the subject an effective amount of a formulation composed of a therapeutically effective amount of a humectant, a therapeutically effective amount of a chelating agent and an effective transport-enhancing amount of a transport enhancer having the formula (I)

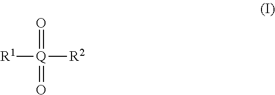

wherein R¹ and R² are independently selected from C₂-C₆ alkyl, C₁-C₆ heteroalkyl, C₆-C₁₄ aralkyl, and C₂-C₁₂ heteroaralkyl, any of which may be substituted, and Q is S or P.

The transport enhancing agent can be, for example, methylsulfonylmethane (MSM; also referred to as methylsulfone, dimethylsulfone, and DMSO₂).

The chelating agent can be selected from ethylenediamine tetraacetic acid (EDTA), ethylene glycol tetraacetic acid (EGTA), cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ArPA), citric acid, acetic acid, phosporic acid, pyro phosphates, meta phosphates, malic acid polymers, etc., and acceptable salts thereof, and any combinations thereof.

These and other aspects will become apparent from the following description of the preferred embodiment taken in conjunction with the following drawings, although variations and modifications therein may be affected without departing from the spirit and scope of the novel concepts of the disclosure.

### DETAILED DESCRIPTION OF THE INVENTION

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term are the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

When referring to a formulation component, it is intended that the term used, e.g., “agent,” encompasses not only the specified molecular entity but also its pharmaceutically acceptable analogs, including, but not limited to, salts, esters, amides, prodrugs, conjugates, active metabolites, and other such derivatives, analogs, and related compounds.

The terms “treating” and “treatment” as used herein refers to the administration of an agent or formulation to a clinically symptomatic individual human or animal afflicted with an adverse condition, disorder, or disease, so as to affect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause. Unless otherwise indicated herein, either explicitly or by implication, if the term “treatment” (or “treating”) is used without reference to possible prevention, it is intended that prevention be encompassed as well, such that “a method for the treatment of gingivitis” would be interpreted as encompassing “a method for the prevention of gingivitis.”

“Optional” or “optionally present”—as in an “optional substituent” or an “optionally present additive” means that the subsequently described component (e.g., substituent or additive) may or may not be present, so that the description includes instances where the component is present and instances where it is not.

By “pharmaceutically acceptable” is meant a material that is not biologically or otherwise undesirable, e.g., the material may be incorporated into a formulation of the invention without causing any undesirable biological effects or interacting in a deleterious manner with any of the other components of the dosage form formulation. However, when the term “pharmaceutically acceptable” is used to refer to a pharmaceutical excipient, it is implied that the excipient has met the required standards of toxicological and manufacturing testing and/or that it is included on the Inactive Ingredient Guide prepared by the U.S. Food and Drug Administration. As explained in further detail infra, “pharmacologically active” (or simply “active”) as in a “pharmacologically active” derivative or analog refers to derivative or analog having the same type of pharmacological activity as the parent agent. The terms “treating” and “treatment” as used herein refer to reduction in severity and/or frequency of symptoms, elimination of symptoms and/or underlying cause, prevention of the occurrence of symptoms and/or their underlying cause, and improvement or remediation of an undesirable condition or damage. Thus, for example, “treating” a subject involves prevention of an adverse condition in a susceptible individual as well as treatment of a clinically symptomatic individual by inhibiting or causing regression of the condition. The term “chelating agent” (or “active agent”) refers to any chemical compound, complex or composition that exhibits a desirable effect in the biological context, i.e., when administered to a subject or introduced into cells or tissues in vitro. The term includes pharmaceutically acceptable derivatives of those active agents specifically mentioned herein, including, but not limited to, salts, esters, amides, prodrugs, active metabolites, isomers, analogs, crystalline forms, hydrates, and the like. When the term “chelating agent” is used, or when a particular chelating agent is specifically identified, it is to be understood that pharmaceutically acceptable salts, esters, amides, prodrugs, active metabolites, isomers, analogs, etc. of the agent are intended as well as the agent per se.

By an “effective” amount or a “therapeutically effective” amount of an active agent is meant a nontoxic but sufficient amount of the agent to provide a beneficial effect. The amount of active agent that is “effective” will vary from subject to subject, depending on the age and general condition of the individual, the particular active agent or agents, and the like. Unless otherwise indicated, the term “therapeutically effective” amount as used herein is intended to encompass an amount effective for the prevention of an adverse condition and/or the amelioration of an adverse condition, i.e., in addition to an amount effective for the treatment of an adverse condition.

As will be apparent to those of skill in the art upon reading this invention, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

Unless otherwise indicated, the invention is not limited to specific formulation components, modes of administration, chelating agents, manufacturing processes, or the like, as such may vary.

Unless otherwise defined, all technical and scientific terms used herein have the same meaning as commonly understood by one of ordinary skill in the art to which this invention pertains. In the case of conflict, the present document, including definitions will control.

### Humectant

Xerostomia or dry mouth is a common condition that results from insufficient saliva volume. It is increasingly prevalent in the aging population and is a side-effect of many medications, as well as cancer treatment. Severe cases of xerostomia are often related to salivary gland dysfunction, known as Sjogren's Syndrome.

The lack of moisture and lubrication typically provided by saliva has a range of negative effects on oral tissue (soft tissue) ranging from mild discomfort to extremely painful and infected mouth sores. The persistent discomfort and dryness can also contribute to larger health issues by causing disruption of sleep, and impairing one's ability to talk (socialize, may impact psychological health) and cat (may impact nutrition). Dry buccal tissue is a less effective barrier and more susceptible to penetration by physical irritants such as toxins and carcinogens in foods, beverages and tobacco. There remains a need for compositions which provide improved relief for xerostomia.

A humectant is a hygroscopic substance which attracts and retains the moisture from the surroundings via absorption, drawing the water vapor into the surface. Commonly used humectants include: commonly used. The most common include: Hyaluronic acid: This is a molecule found naturally in the body, although the body makes less of it with age. Glycerin: Also called glycerine or glycerol, glycerin is a clear substance that occurs naturally in animal and plant tissue, including that of humans. A lot of the glycerin in makeup is made from soybean and sugarcane. Alpha hydroxy acids (AHA): Alpha hydroxy acids like glycolic acid, lactic acid, and citric acid are naturally derived acids from fruit and milk sugars. Along with drawing in water, glycolic acid comes from sugar cane, lactic acid from milk, and citric acid from citrus fruits.

Oral care formulations of the invention include one or more humectants. Illustrative useful humectants can include, for example glycerol, sorbitol, xylitol, maltitol, propylene glycol, hexylene glycol, butylene glycol, erythritol, isomalt or combinations thereof. Humectants such as xylitol, erythritol, or combinations thereof may be especially useful because they would provide benefits to the hard tissues of the mouth as well as provide humectant properties. In disclosed articles, the one or more humectant can be present in an amount of not less than 5 percent based on the total weight of the article, or not less than 7 percent based on the total weight of the article. In disclosed articles, the one or more humectant can be present in an amount of not greater than 15 percent based on the total weight of the article, or not greater than 12 percent based on the total weight of the article.

Humectants can enhance the viscosity, mouthfeel, and sweetness of the product, and may also help preserve the product from degradation or microbial contamination. Suitable humectants include edible polyhydric alcohols such as glycerin, sorbitol, xylitol, propylene glycol as well as other polyols and mixtures of these humectants. Sorbitol may in some cases be provided as a hydrogenated starch hydrolysate in syrup form, which comprises primarily sorbitol (the product if the starch were completely hydrolyzed to glucose, then hydrogenated), but due to incomplete hydrolysis and/or presence of saccharides other than glucose, may also include other sugar alcohols such mannitol, maltitol, and longer chain hydrogenated saccharides, and these other sugar alcohols also function as humectants in this case.

In some embodiments, an oral care composition for use in accordance with the present disclosure comprises an orally acceptable humectant including, without limitation, polyhydric alcohols such as glycerin, sorbitol, xylitol or low molecular weight PEGs. Several humectants also function as sweeteners. One or more humectants are optionally present in a total amount of 1 weight % to 50 weight % by total weight of the composition. The one or more humectant is selected from glycerol, sorbitol, xylitol, maltitol, propylene glycol, hexylene glycol, butylene glycol, erythritol, isomalt, and a combination thereof.

### Transport Enhancer

The transport enhancer is selected to facilitate the transport of a chelating agent through the tissues, extra-cellular matrices, and/or cell membranes of a body. An “effective amount” of the transport enhancer represents an amount and concentration within a formulation of the invention that is sufficient to provide a measurable increase in the penetration of a chelating agent through one or more of the sites of oral cavity or teeth in a subject than would otherwise be the case without the inclusion of the transport enhancer within the formulation.

The concentration of the transport enhancer must be sufficient to allow the chelating agent to be transferred across the cell membrane. Thus, the concentration or relative amount of the transport enhancer will be decided by the thickness of the cell membrane in the animal or human subject who is ingesting the formulation. In some embodiments, the concentration of MSM in the present formulations is in the range of about 0.0001 wt. % to 30 wt. %, or from about 0.01 wt. % to about 0.10, 1, 5, 10, 20, 30 wt. %, and preferably between about 0.01 wt. % to 1.0 wt. %.

The transport enhancer is generally of the formula (I)

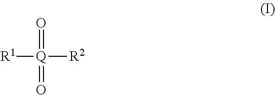

wherein R¹ and R² are independently selected from C₂-C₆ alkyl, C₁-C₆ heteroalkyl, C₆-C₁₄ aralkyl, and C2-C12 heteroaralkyl, any of which may be substituted, and Q is S or P. Compounds wherein Q is S and R¹ and R² are C₁-C₃ alkyl are preferred, with methylsulfonylmethane (MSM) being the optimal transport enhancer.

The phrase “having the formula” or “having the structure” is not intended to be limiting and is used in the same way that the term “comprising” is commonly used. With respect to the above structure, the term “alkyl” refers to a linear, branched, or cyclic saturated hydrocarbon group containing 1 to 6 carbon atoms, such as methyl, ethyl, n-propyl, isopropyl, n-butyl, isobutyl, t-butyl, cyclopentyl, cyclohexyl and the like. If not otherwise indicated, the term “alkyl” includes unsubstituted and substituted alkyl, wherein the substituents may be, for example, halo, hydroxyl, sulfhydryl, alkoxy, acyl, etc. The term “alkoxy” intends an alkyl group bound through a single, terminal ether linkage; that is, an “alkoxy” group may be represented as —O-alkyl where alkyl is as defined above. The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Aryl” includes unsubstituted and substituted aryl, wherein the substituents may be as set forth above with respect to optionally substituted “alkyl” groups. The term “aralkyl” refers to an alkyl group with an aryl substituent, wherein “aryl” and “alkyl” are as defined above. Preferred aralkyl groups contain 6 to 14 carbon atoms, and particularly preferred aralkyl groups contain 6 to 8 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The term “acyl” refers to substituents having the formula —(CO)-alkyl, —(CO)-aryl, or —(CO)-aralkyl, wherein “alkyl,” “aryl,” and “aralkyl” are as defined above. The terms “heteroalkyl” and “heteroaralkyl” are used to refer to heteroatom-containing alkyl and aralkyl groups, respectively, i.e., alkyl and aralkyl groups in which one or more carbon atoms is replaced with an atom other than carbon, e.g., nitrogen, oxygen, sulfur, phosphorus or silicon, typically nitrogen, oxygen or sulfur.

The formulation also includes an effective amount of a transport enhancer that facilitates penetration of the formulation components through cell membranes, tissues, and extracellular matrices. The “effective amount” of the transport enhancer represents a concentration that is sufficient to provide a measurable increase in penetration of one or more of the formulation components through membranes, tissues, and extracellular matrices as just described. Suitable transport enhancers include, by way of example, methylsulfonylmethane (MSM; also referred to as methyl sulfone), combinations of MSM with dimethylsulfoxide (DMSO), or a combination of MSM and, in a less preferred embodiment, DMSO, with MSM particularly preferred. DMSO, a transport enhancer but essentially a solvent, is not particularly suitable for formulations according to this invention. DMSO works as a highly potent solvent and therefore a carrier of its solutes. In contrast, MSM works in a totally different manner by forming hydrogen bonds with select molecules and changing their charge characteristics of the target molecule allowing the target molecule to get through charged barriers like biologic membranes.

There are differences in chemical structures between MSM and DMSO. Methylsulfonylmethane (MSM) is an organosulfur compound with the formula (CH₃)₂SO₂. It is also known by several other names including DMSO₂, methyl sulfone, and dimethyl sulfone. This colorless solid feature a sulfonyl functional group and is considered relatively inert chemically. MSM has the structure:

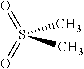

Dimethyl sulfoxide (DMSO) on the other hand is an organosulfur compound with the formula (CH₃)₂SO. This colorless liquid is a widely-used polar aprotic solvent that dissolves both polar and nonpolar compounds and is miscible in a wide range of organic solvents as well as water. DMSO has the structure:

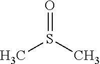

MSM is an odorless, highly water-soluble (34% w/v @ 79° F.) white crystalline compound with a melting point of 108-110° C. and a molecular weight of 94.1 g/mol. MSM serves as a multifunctional agent herein, insofar as the agent not only increases cell membrane permeability but also acts as a “transport facilitating agent” (TFA) that aids in the transport of one or more formulation components to oral tissues. Furthermore, MSM per se provides medicating effects and can serve as an anti-inflammatory agent as well as an analgesic. MSM also acts to improve oxidative metabolism in biological tissues and is a source of organic sulfur, which assists in the reduction of scarring. MSM additionally possesses unique and beneficial solubilizing properties, in that it is soluble in water, as noted above, but exhibits both hydrophilic and hydrophobic properties because of the presence of polar S=O groups and nonpolar methyl groups. The molecular structure of MSM also allows for hydrogen bonding with other molecules, i.e., between the oxygen atom of each S=O group and hydrogen atoms of other molecules, and for the formation of van der Waal associations, i.e., between the methyl groups and nonpolar (e.g., hydrocarbon) segments of other molecules.

### Chelating Agent

Chelation is a chemical combination with a metal in complexes in which the metal is part of a ring. An organic ligand is called a chelator or chelating agent, and the chelate is a metal complex. The larger the number of ring closures to a metal atom, the more stable the compound. The stability of a chelate is also related to the number of atoms in the chelate ring. Monodentate ligands that have one coordinating atom, like H₂O or NH₃, are easily broken apart by other chemical processes, whereas polydentate chelators, donating multiple binds to metal ions, provide more stable complexes. Chlorophyll, a green plant pigment, is a chelate that consists of a central magnesium atom joined with four complex chelating agents (pyrrole ring). Heme is an iron chelate that contains iron (II) ions in the center of the porphyrin. Chelating agents offer a wide range of sequestrants to control metal ions in aqueous systems. By forming stable water-soluble complexes with multivalent metal ions, chelating agents prevent undesired interaction by blocking the normal reactivity of metal ions. EDTA (ethylenediamine tetraacetate) is a good example of a common chelating agent which have nitrogen atoms and short-chain carboxylic groups.

For the purposes of this invention that is safely ingestible is suitable for formulating a composition that can be consumed by a subject in need of iron chelation. Examples of chelators of iron and calcium include but are not limited to, diethylene triamine pentaacetic acid (DTPA), ethylene diamine tetraacetic acid (EDTA), nitrilotriacetic acid (NTA), 1,3-propylene diamine tetraacetic acid (PDTA), Ethylene diamine disuccinic acid (EDDS), and ethylene glycol tetraacetic acid (EGTA). Any suitable chelating agent known in the art, which is biologically safe and can chelate iron, calcium, or other metals, is suitable for the invention.

The concentration of the chelator must be sufficient to allow the chelating agent to be transferred across the cell membrane in order to access intracellular iron. Thus, the concentration of the chelator will be decided by the thickness of the cell membrane in the animal or human subject who is ingesting the formulation. For example, in exemplary evaluations, rats were dosed with ad libitum drinking water with 26 ppm (0.0026%) EDTA disodium which, adjusted for humans, would be equivalent to 260 ppm (0.026%). In some embodiments, the transport enhancer may be present in a formulation of the invention in an amount that ranges from about 0.0001 wt. % to about 15 wt. %, typically in the range of about 0.001 wt. % to about 1 wt. %, more typically in the range of about 0.10 wt. % to about 5 wt. %.

Compounds useful as chelating agents herein include any compounds that coordinate to or form complexes with a divalent or polyvalent metal cation, thus serving as a sequestrant of such cations. Accordingly, the term “chelating agent” herein includes not only divalent and polyvalent ligands (which are typically referred to as “chelators”) but also monovalent ligands capable of coordinating to or forming complexes with the metal cation.

Suitable biocompatible chelating agents useful in conjunction with the present invention include, without limitation, monomeric polyacids such as EDTA, cyclohexanediamine tetraacetic acid (CDTA), N-(2-Hydroxyethyl) ethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, pharmaceutically acceptable salts thereof, and combinations of any of the foregoing. Other exemplary chelating agents include: phosphates, e.g., pyrophosphates, tripolyphosphates, and hexametaphosphates, malic acid polymers, and others.

EDTA and acceptable EDTA salts are particularly preferred, wherein representative acceptable EDTA salts are typically selected from diammonium EDTA, disodium EDTA, dipotassium EDTA, triammonium EDTA, trisodium EDTA, tripotassium EDTA, and calcium disodium EDTA.

EDTA has been widely used as an agent for chelating metals in biological tissue and blood and has been suggested for inclusion in various formulations. For example, U.S. Pat. No. 6,348,508 to Denick Jr. et al. describes EDTA as a sequestering agent to bind metal ions. In addition to its use as a chelating agent, EDTA has also been widely used as a preservative in place of benzalkonium chloride, as described, for example, in U.S. Pat. No. 6,211,238 to Castillo et al. U.S. Pat. No. 6,265,444 to Bowman et al. discloses use of EDTA as a preservative and stabilizer. However, EDTA has generally not been applied topically in any significant concentration formulations because of its poor penetration across biological membranes.

Among the chelating/sequestering materials which may be included in the compositions there may be mentioned biocompatible chelating agents include, without limitation, monomeric polyacids such as EDTA, cyclohexanediamine tetraacetic acid (CDTA), N-(2-Hydroxyethyl) ethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, pharmaceutically acceptable salts thereof, and combinations of any of the foregoing.

Other exemplary chelating agents include: phosphates, e.g., pyrophosphates, tripolyphosphates, and hexametaphosphates. Other exemplary chelating agents include: phosphates, e.g., pyrophosphates, tripolyphosphates, and hexametaphosphates; chelating antibiotics such as chloroquine and tetracycline; nitrogen-containing chelating agents containing two or more chelating nitrogen atoms within an imino group or in an aromatic ring (e.g., diimines, 2,2′-bipyridines, etc.); and polyamines such as cyclam (1,4,7,11-tetraazacyclotetradecane), N-(C₁-C₃₀ alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), deferoxamine (N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide, or N′-[5-(Acetyl-hydroxy-amino)pentyl]-N-[5-[3-(5-aminopentyl-hydroxy-carbamoyl) propanoylamino]pentyl]-N-hydroxy-butane diamide); also known as desferrioxamine B, desferoxamine B, DFO-B, DFOA, DFB or desferal), deferiprone, pyridoxal isonicotinoyl hydrazone (PIH), salicylaldehyde isonicotinoyl hydrazone (SIH), ethane-1,2-bis(N-1-amino-3-ethylbutyl-3-thiol).

Additional, suitable biocompatible chelating agents which may be useful for the practice of the current disclosure include EDTA-4-aminoquinoline conjugates such as ([2-(Bis-ethoxycarbonylmethyl-amino)-ethyl]-{[2-(7-chloro-quinolin-4-ylamino)-ethylcarbamoyl]-methyl}-amino)-acetic acid ethyl ester, ([2-(Bis-ethoxycarbonylmethyl amino)-propyl]-{[2-(7-chloro-quinolin-4-ylamino)-ethylcarbamoyl]-methyl}-amino)-acetic acid ethyl ester, ([3-(Bis-ethoxycarbonylmethyl-amino)-propyl]-{[2-(7-chloro-quinolin-4-ylamino)-ethylcarbamoyl]-methyl}-amino)-acetic acid ethyl ester, ([4-(Bis-ethoxycarbonylmethyl-amino)-butyl]-{[2-(7-chloro-quinolin-4-ylamino)-ethylcarbamoyl]-methyl}-amino)-acetic acid ethyl ester, ([2-(Bis-ethoxymethyl-amino)-ethyl]-{[2-(7-chloro-quinolin-4-ylamino)-ethylcarbamoyl]-methyl}-amino)-acetic acid ethyl ester, ([2-(Bis-ethoxymethyl-amino)-propyl]-{[2-(7-chloro-quinolin-4-ylamino)-ethylcarbamoyl]-methyl}-amino)-acetic acid ethyl ester, ([3-(Bis-ethoxymethyl-amino)-propyl]-{[2-(7-chloro-quinolin-4-ylamino)-ethylcarbamoyl]-methyl}-amino)-acetic acid ethyl ester, ([4-(Bis-ethoxymethyl-amino)-butyl]{[2-(7-chloro-quinolin-4-ylamino)-ethylcarbamoyl]-methyl}-amino)-acetic acid ethyl ester as described in Solomon et al., Med. Chem. 2:133-138, 2006.

Additionally, natural chelators include, but are not limited to, citric acid, phytic acid, lactic acid, acetic acid and their salts. Other natural chelators and weak chelators include but are not limited to curcumin (turmeric), ascorbic acid, succinic acid, and the like.

In some embodiments, the chelating agents are selected from the tetrasodium salt of iminodisuccinic acid (BAYPURE® CX100; LANXESS GMBH (previously Bayer Chemicals) Leverkusen, DE) or salts of poly-aspartic acid (PASA—BAYPURE® DS100; LANXESS GMBH, Leverkusen, DE). In some embodiments, the chelating agents are tetra sodium salts of L-glutamic acid N,N-diacetic acid (GLDA—DISSOLVINE®, AkzoNobel, Netherlands).

In some embodiments, the chelating agent incorporated in the formulation is a prochelator. A prochelator is any molecule that is converted to a chelator when exposed to the appropriate chemical or physical conditions. For example, BSIH (isonicotinic acid [2-(4,4,5,5-tetramethyl-[1,3,2] dioxaborolan-2-yl)-benzylidene]-hydrazide) prochelators are converted by hydrogen peroxide into SIH (salicylaldehyde isonicotinoyl hydrazone) iron-chelating agents that inhibit iron-catalyzed hydroxyl radical generation.

The inactivated metal ion sequestering agent is sometimes referred to herein as a “prochelator,” although sequestration of metal ions can involve sequestration and complexation processes beyond the scope of chelation per se. The term “prochelator” is analogous to the term “prodrug” insofar as a prodrug is a therapeutically inactive agent until activated in vivo, and the prochelator, as well, is incapable of sequestering metal ions until activated in vivo.

### Formulations

A variety of means can be used to formulate the compositions of the invention. Techniques for formulation and administration may be found in “Remington: The Science and Practice of Pharmacy,” Twenty Third Edition, Adeboye Adejare, editor-in-chief; Academic Press; 23rd edition (Nov. 13, 2020). For human or animal administration, preparations should meet sterility, pyrogenicity, general safety and purity standards comparable to those required by the FDA. Administration of the pharmaceutical formulation can be performed in a variety of ways, some of which are as described herein.

Other possible additives for incorporation into the formulations that are at least partially aqueous include, without limitation, thickeners, isotonic agents, buffering agents, and preservatives, providing that any such excipients do not interact in an adverse manner with any of the formulation's other components. It should also be noted that preservatives are not generally necessarily in light of the fact that the selected chelating agent itself serves as a preservative.

The chelator and the permeation agent are dissolved in a solvent selected from a non-limiting list of solvents that may be employed: water, ethanol, acetone, DMSO, isopropanol, glycerol, propylene glycol, polyethylene glycol, propylene carbonate, and ethyl acetate.

The oral care composition used in the present disclosure comprise significant levels of water. Water employed in the preparation of commercial oral compositions should be deionized and free of organic impurities. The amount of water in the compositions includes the free water that is added plus that amount which is introduced with other materials.

Mouthwashes frequently contain significant levels of ethanol, which is often needed to solubilize essential oils and to prevent bacterial contamination. High levels of ethanol may be undesirable, because in addition to the potential for abuse by ingestion, the ethanol may exacerbate conditions like xerostomia. Accordingly, in some embodiments, the oral care compositions of the invention are substantially free of ethanol, e.g., contain less than 1% ethanol or less than 0.1% ethanol.

In some embodiments, the oral care composition is a dentifrice comprising a humectant, e.g., selected from glycerin, sorbitol, propylene glycol, polyethylene glycol, xylitol, and mixtures thereof, e.g., comprising at least 30%, e.g., 40-50% glycerin, by weight of the composition.

In some embodiments, the formulation further comprises an emulsifier, wherein the emulsifier is selected from the group consisting of gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono- and di-glycerides, propylene glycol monoesters, polyglycerol esters, diacetylated tartaric acid esters of mono-and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.

In some embodiments, the beverage formulation further comprises a flavoring agent is selected from the group consisting of vanilla, vanillin, ethyl vanillin, orange oil, peppermint oil, strawberry, raspberry, and mixtures thereof. The flavoring could also be other synthetic or natural flavors or a combination thereof.

### EXAMPLES

The following examples are put forth so as to provide those skilled in the art with a complete invention and description of how to make and use embodiments in accordance with the invention and are not intended to limit the scope of what the inventors regard as their discovery. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Measurement of Xerostomia

Physical examination is focused on the oral cavity, specifically any apparent dryness (eg, whether the mucosa is dry, sticky, or moist; whether saliva is foamy, thick, stringy, or normal in appearance), the presence of any lesions caused by Candida albicans, and the condition of the teeth.

The presence and severity of xerostomia can be assessed in several ways. For example, a tongue blade can be held against the buccal mucosa for 10 seconds. If the tongue blade falls off immediately when released, salivary flow is considered normal. The more difficulty encountered removing the tongue blade, the more severe the xerostomia. In women, the lipstick sign, where lipstick adheres to the front teeth, may be a useful indicator of xerostomia.

If there appears to be dryness, the submandibular, sublingual, and parotid glands should be palpated while observing the ductal openings for saliva flow. The openings are at the base of the tongue anteriorly for the submandibular and sublingual glands and on the middle of the inside of the check for the parotid glands. Drying the duct openings with a gauze square before palpation aids observation. If a graduated container is available, the patient can expectorate once to empty the mouth and then expectorate all saliva into the container. Normal production is 0.3 to 0.4 mL/min. Significant xerostomia is 0.1 mL/min.

For patients in whom the presence of xerostomia is unclear, sialometry can be conducted by placing collection devices over the major duct orifices and then stimulating salivary production with citric acid or by chewing paraffin. Normal parotid flow is 0.4 to 1.5 mL/min/gland. Flow monitoring can also help determine response to therapy.

### Clinical Evaluation I: Effect of LivFresh Dental Gel with Propylene Glycol on Saliva Production in Individuals with Mild to Moderate Xerostomia (Dry Mouth)

This study aimed to quantify the effect over 2 weeks of a test dentifrice (LivFresh Dental Gel plus Propylene Glycol (Livionex, Los Gatos, CA, USA)) on unstimulated and stimulated saliva production as well as oral comfort in 4 individuals with mild to moderate xerostomia.

OVERVIEW: Baseline unstimulated and stimulated saliva production over 5 minutes was measured on 3 subsequent days. Then subjects switched from their regular (non-Livionex) dentifrice to brushing twice daily for 2 minutes with LIVFRESH® Dental Gel plus Propylene Glycol (Livionex, Los Gatos, CA, USA). Unstimulated and stimulated saliva was collected over 5 minutes on Days 1, 2, 3, 7 and 14, and subjects completed an open-ended questionnaire with regard to the effects of the dental gel on their dry mouth and overall oral comfort at each visit.

MATERIALS AND METHODS: This study was performed under UCI approved IRB protocol #2013-9778 and all clinical procedures were conducted in accordance with the Helsinki Declaration of 1975, as updated in 2013. No significant changes were made in the study design after commencement of the study. The rolling recruitment through advertisements was initiated in December 2021 and all the study visits were completed by a single examiner by end of January 2022.

SUBJECTS: Four patients were enrolled in this study. They all met the following inclusion and exclusion criteria:

Inclusion Criteria:

1. Male or non-pregnant female, 18 years of age and above.

2. A minimum of 25 teeth are present.

3. Willing and able to provide written informed consent.

4. Willing and able to comply with study visits as described in the protocol.

5. Available for follow-up on the telephone.

6. Mild to moderate xerostomia as diagnosed by their dentist

Exclusion Criteria:

1. Unable or unwilling to sign the informed consent form.

2. Participation in any other clinical study within the last 30 days prior to enrollment into this study.

3. Subjects who must receive dental treatment during the study duration.

4. History of significant adverse effects following the use of oral hygiene products such as toothpaste and mouth rinses.

5. Allergy to personal care/consumer products or their ingredients.

6. In the last 3 months, anti-inflammatory drugs, or immune suppressants.

7. Presence of any condition, abnormality, or situation at the Baseline that in the opinion of the Principal Investigator may affect the patient's ability to comply with study requirements.

STUDY DENTIFRICES: Test dentifrice used: LIVFRESH Dental Gel®—Updated Formulation (Livionex, Los Gatos, CA, USA).

TABLE 1
List of components of test dentifrice and corresponding functions

| Ingredient | Function |
|---|---|
| EDTA, disodium dihydrate | Plaque control agent |
| Methyl Sulfonyl Methane | Transport Agent to allow EDTA |
| (MSM) | to access dental plaque |
| ι-Carrageenan gum | Gelling Agent |
| Konjac Gum | Thickening Agent |
| Stevia | Sweetener |
| Sodium Hydroxide | pH Adjustor |
| Flavors (Menthol, Menthyl | Flavoring Agents |
| Lactate, Peppermint Oil) | |
| Sunflower Lecithin | Emulsifier for peppermint oil |
| FD&C Blue #1 6.5% solution | Food coloring |
| Water | Diluent |

TEST PROTOCOL

This single-center study was designed as a prospective study. Subjects underwent baseline unstimulated and stimulated 5-minute saliva collection using standard technique (please see below) on 3 subsequent days, then their usual oral hygiene regimen was replaced by brushing for 2 minutes twice daily with the test dentifrice over a period of 14 days. In addition to the 3 baseline days, saliva was collected on days 1, 2, 3, 7, and 14. At each visit, subjects completed an open-ended questionnaire concerning the perceived dryness and overall feeling of their mouth. Saliva was collected in standard tightly sealed containers, labeled, and stored in a refrigerator in sealed bags. All samples were weighed at the study end on the same laboratory scales accurate to 0.0002 g (Ohaus EX324N Explorer Analytical Balance).

Saliva collection was performed according to the University of Southern California School of Dentistry guidelines for saliva collection (https://jada(dot)ada(dot)org/article/S0002-8177(14)63880-0/pdf).

COLLECTION OF UNSTIMULATED WHOLE SALIVA: The patient is advised to refrain from intake of any food or beverage (water exempted) one hour before the test session. Smoking, chewing gum and intake of coffee also are prohibited during this hour. The subject is advised to rinse his or her mouth several times with deionized (distilled) water and then to relax for five minutes.

COLLECTION OF STIMULATED WHOLE SALIVA-GUM: Saliva collection was according to the following protocol:

1. Instruct the subject to sit motionless.

2. Instruct the subject to lean the head forward over the funnel.

3. Instruct the subject to swallow to void the mouth of saliva (starting time).

4. Instruct the subject to chew the inert gum base according to the pace of the metronome (approximately 70 strokes per minute).

5. At one-minute intervals, ask the subject to spit saliva into the tube without swallowing. Tell the subject, “Spit out, keep chewing” (after the first minute), “Spit out, keep chewing” (after the second minute), etc. Discard the first two-minute collection. A plastic or paper cup may be used for this collection. Proceed with another 5-minute collection. Save this sample for further analysis if indicated.

6. Ask the patient to spit everything (that is, both saliva and gum base) into the tube.

7. Remove the gum base from the funnel before weighing the tube and funnel with saliva.

EVALUATION CRITERIA: Saliva samples were weighed using calibrated laboratory scales with an accuracy of 0.0002g (Ohaus EX324N Explorer Analytical Balance). Subjects' comments were recorded in a dedicated notebook

STATISTICAL ANALYSIS: Sample weights were described using means and standard deviations (SDs). Change over time for the digital, continuous weight scale was analyzed using repeated measures analysis of variance (ANOVA) models.

SALIVA COLLECTION:

TABLE 2
Mean saliva collected in 5 minutes

| | Mean Saliva Weight (S.D.) (g) | | Saliva Flow Rate per Minute | |
|---|---|---|---|---|
| | Unstimulated | Stimulated | Unstimulated | Stimulated |
| Mean Baseline (n = 24) | 0.79 (0.39) | 9.92 (1.01) | 0.16 | 1.98 |
| Day 1 (n = 8) | 0.92 (0.36) | 7.98 (0.84) | 0.18 | 1.60 |
| Day 2 (n = 8) | 0.91 (0.53) | 6.12 (0.42) | 0.18 | 1.22 |
| Day 3 (n = 8) | 2.55 (1.44) | 8.21 (0.54) | 0.51 | 1.64 |
| Day 7 (n = 8) | 3.23 (1.44) | 12.32 (1.70) | 0.65 | 2.46 |
| Day 14 (n = 8) | 6.24 (0.65) | 14.76 (1.14) | 1.25 | 2.95 |

Unstimulated (p = 0.0045) and stimulated (p = 0.00031) saliva collection increased significantly over the study duration.

The mean unstimulated saliva flow rate (which more closely correlates with hyposalivation) was 0.16 ml/min at baseline and brushing with the test dental gel increased by 7.9 times to 1.25 ml/min on Day 14. Unstimulated saliva flow rates between 0.10 to 0.25 ml/min are generally considered low or have moderate hyposalivation.

This study demonstrated considerable and highly significant increases in unstimulated and stimulated saliva production after two weeks of toothbrushing twice daily with the test dental gel. These quantitative data were strongly supported by the comments of the subjects, who all stated repeatedly that their mouths felt better and cleaner overall, and specifically, their feeling of dry mouth was alleviated by brushing with the test dental gel.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claim.

What is claimed is:

1. A formulation, comprising:
a humectant;
a permeation enhancer that is methylsulfonylmethane (MSM);
one or more inert excipients; and
a liquid vehicle or carrier;
wherein the humectant and the permeation enhancer are present in proportions effective to treat one or more symptoms of xerostomia and/or alleviate dry mouth, and
wherein the percentage of chelator is about 0.05% to 15% and the percentage of permeation enhancer in the composition is about 0.0001% to 30% by weight, respectively.

2. The formulation of claim 1, wherein the xerostomia is associated with a condition or disorder selected from: Sjögren's syndrome, diabetes mellitus, rheumatoid arthritis, systemic lupus erythematosus, head and neck injuries resulting in damage to nerves of the salivary glands, radiation therapy and chemotherapy of head and neck, tobacco use, and methamphetamine use.

3. The formulation of claim 1, wherein the xerostomia is a side effect associated with use of medication.

4. The formulation of claim 1, wherein the composition is selected from a toothpaste, a gel dentifrice, a tooth powder, a mouth-rinse, a mouthwash, a tooth hardener, an anti-calculus composition, a gum, a lozenge or a format suitable applying the composition to an oral surface, teeth, or gums.

5. The formulation of claim 1, wherein the humectant is selected from one or more of glycerol, sorbitol, xylitol, maltitol, propylene glycol, hexylene glycol, butylene glycol, erythritol, isomalt, and a combination thereof.

6. The formulation of claim 4, wherein the humectant is propylene glycol.

7. The formulation of claim 1, wherein the chelating agent is selected from ethylenediamine tetraacetic acid (EDTA), ethylene glycol tetraacetic acid (EGTA), cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ArPA), citric acid, acetic acid and acceptable salts thereof, and any combinations thereof.

8. The formulation of claim 12, wherein the EDTA salt is selected from diammonium EDTA, disodium EDTA, dipotassium EDTA, triammonium EDTA, trisodium EDTA, tripotassium EDTA, tetrasodium EDTA, tetrapotassium EDTA, calcium disodium EDTA, and combinations thereof.

9. The formulation of claim 1, wherein the chelating agent is selected from phosphates, pyrophosphates, tripolyphosphates, and hexametaphosphates.

10. The formulation of claim 1, wherein the chelating agent is a nitrogen-containing chelating agents containing two or more chelating nitrogen atoms within an imino group or in an aromatic ring, diimines, or 2,2′-bipyridines.

11. The formulation of claim 1, wherein the chelating agent is a polyamine selected from cyclam (1,4,7,11-tetraazacyclotetradecane), N-(C₁-C₃₀ alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), deferoxamine (N′-{5-[Acetyl(hydroxy)amino]pentyl}-N-[5-({4- (5-aminopentyl)(hydroxy)amino]-4-oxobutanoyl}amino)pentyl]-N-hydroxysuccinamide, or N′-[5-(Acetyl-hydroxy-amino) pentyl]-N-[5-[3-(5-aminopentyl-hydroxy-carbamoyl) propanoylamino] pentyl]-N-hydroxy-butane diamide), desferrioxamine B, desferoxamine B, DFO-B, DFOA, DFB, desferal, deferiprone, pyridoxal isonicotinoyl hydrazone (PIH), salicylaldehyde isonicotinoyl hydrazone (SIH), ethane-1,2-bis (N-1-amino-3-ethylbutyl-3-thiol).

12. The formulation of claim 1, wherein the chelating agent is a EDTA-4-aminoquinoline conjugate selected from ([2-(Bis-ethoxycarbonylmethyl-amino)-ethyl]- {[2-(7-chloro-quinolin-4-ylamino)-ethylcarbamoyl]-methyl}-amino)-acetic acid ethyl ester, ([2-(Bis-ethoxycarbonylmethyl-amino)-propyl]-{[2-(7-chloro-quinolin-4-ylamino)-ethylcarbamoyl]-methyl}-amino)-acetic acid ethyl ester, ([3-(Bis-ethoxycarbonylmethyl-amino)-propyl]- {[2-(7-chloro-quinolin-4-ylamino)-ethylcarbamoyl]-methyl}-amino)-acetic acid ethyl ester, ([4- (Bis-ethoxycarbonylmethyl-amino)-butyl]-{[2-(7-chloro-quinolin-4-ylamino)-ethylcarbamoyl]-methyl}-amino)-acetic acid ethyl ester, ([2-(Bis-ethoxymethyl-amino)-ethyl]-{[2-(7-chloro-quinolin-4-ylamino)-ethylcarbamoyl]-methyl}-amino)-acetic acid ethyl ester, ([2-(Bis-ethoxymethyl-amino)-propyl]-{[2-(7-chloro-quinolin-4-ylamino)-ethylcarbamoyl]-methyl}-amino)-acetic acid ethyl ester, ([3-(Bis-ethoxymethyl-amino)-propyl]-{[2-(7-chloro-quinolin-4-ylamino)-ethylcarbamoyl]-methyl}-amino)-acetic acid ethyl ester, ([4-(Bis-ethoxymethyl-amino)-butyl]-{[2-(7-chloro-quinolin-4-ylamino)-ethylcarbamoyl]-methyl}-amino)-acetic acid ethyl ester.

13. The formulation of claim 1, wherein the chelating agent is a tetrasodium salt of iminodisuccinic acid.

14. The formulation of claim 1, wherein the chelating agent is poly-aspartic acid or a salt thereof.

15. The formulation of claim 1, wherein the chelating agent is a tetrasodium salt of L-glutamic acid N,N-diacetic acid.

16. The formulation of claim 1, wherein the chelating agent is a natural chelator selected from citric acid, phytic acid, lactic acid, acetic acid and their salts, and curcumin.

17. The formulation of claim 1, wherein the formulation further comprises an emulsifier, wherein the emulsifier is selected from the group consisting of gum arabic, modified starch, pectin, xanthan gum, gum ghatti, gum tragacanth, fenugreek gum, mesquite gum, mono-glycerides and di-glycerides of long chain fatty acids, sucrose monoesters, sorbitan esters, polyethoxylated glycerols, stearic acid, palmitic acid, mono-glycerides, di-glycerides, propylene glycol esters, lecithin, lactylated mono-and di-glycerides, propylene glycol monoesters, polyglycerol esters, diacetylated tartaric acid esters of mono-and di-glycerides, citric acid esters of monoglycerides, stearoyl-2-lactylates, polysorbates, succinylated monoglycerides, acetylated monoglycerides, ethoxylated monoglycerides, quillaia, whey protein isolate, casein, soy protein, vegetable protein, pullulan, sodium alginate, guar gum, locust bean gum, tragacanth gum, tamarind gum, carrageenan, furcellaran, Gellan gum, psyllium, curdlan, konjac mannan, agar, and cellulose derivatives, or combinations thereof.

18. The formulation of claim 1, wherein the chelator is at a concentration of 0.002% to 0.1% w/v.

19. The formulation of claim 1, wherein the MSM is at a concentration of 0.005% to 3% w/v.

20. The formulation of claim 1, wherein the humectant is at a concentration of 0.05% to 15% w/v.

21. The formulation of claim 1, further comprising isotonic agents selected from sugars, buffers and sodium chloride.

22. The formulation of claim 1, further comprising a buffering agent.

23. The formulation according to claim 1, wherein the composition can affect hydration loss in an area contacted by the composition.

24. The formulation according to claim 1, wherein the composition can increase lubricity or lubriciousness of an area contacted by the composition.

25. A method of decreasing hydration loss in an oral tissue, the method comprising: contacting an oral tissue with a composition according to any of claims 1-24.

26. A method of increasing lubricity or lubriciousness in an oral tissue, the method comprising: contacting an oral tissue with a composition according to any of claims 1-24.

27. A method of alleviating the effects of xerostomia, dry mouth, or both, the method comprising: contacting an oral tissue with a composition according to any of claims 1-24.
